(19) United States
(12) Patent Application Publication
(10) Pub. No.: US 2009/0202496 A1
(43) Pub. Date: Aug. 13, 2009

(54) METHOD FOR REGENERATING AN IMMUNE SYSTEM

(75) Inventors: Mitchell J. Ghen, Hillsboro Beach, FL (US); Ramesh Roshan, West Palm Beach, FL (US); Romin Roshan, Halladale Beach, FL (US)

Correspondence Address: RATNERPRESTIA, P.O. BOX 980, VALLEY FORGE, PA, 19482, US

(73) Assignee: EDEN BIOTECH LTD., Nassau (BS)

(21) Appl. No.: 11/997,069

(22) PCT Filed: Jul. 27, 2006
(86) PCT No.: PCT/US06/29483

§ 371 Date: Jul. 30, 2008

Related U.S. Application Data
(60) Provisional application No. 60/703,073, filed on Jul. 28, 2005.

(51) Int. Cl. A61K 35/14; C12N 5/08; A61P 37/04; A61P 31/18

(52) U.S. Cl. 424/93.7; 435/372

(57) ABSTRACT

An isolated and purified cell line of hematopoeitic stem cells (HSC) that are incapable of expressing the CCR5 receptor on the cell surface (“the CCR5Δ32 cells” are used to regenerate the immune system in a subject in need thereof and especially to treat a subject infected with human immunodeficiency virus (HIV). The method is carried out by transplanting CCR5Δ32 into the recipient subject. Because mature immune cells derived from CCR5Δ32 cells cannot express functional CCR5 receptors, they will be resistant to infection by HIV and other pathogens that use the CCR5 receptor to infect cells. An embodiment of the invention includes administration of a nutritional regimen to the patient that optimizes conditions for CCR5Δ32 cell transplantation. Another embodiment of the invention includes co-transplanting mesenchymal cells along with the HSC in order to enhance the growth and development of the transplanted HSC.

### RELATED APPLICATIONS

This application claims the benefit of priority from U.S. application Ser. No. 60/703,073 filed Jul. 28, 2005, the contents of which are incorporated herein by reference.

### FIELD OF THE INVENTION

This invention relates to regenerating an immune system and particularly to treating a subject infected with human immunodeficiency virus (HIV), and an isolated and purified stem cell line for doing so.

### BACKGROUND OF THE INVENTION

Transplantation of allogeneic or autologous hematopoietic stem cells (HSC) is an established treatment for a variety of hematological diseases and metabolic disorders. After transplantation, the HSC differentiate into functional immune cells and regenerate or reconstitute the previously damaged immune system. However, stem cell transplantation is ineffective in controlling disease if the regenerated immune cells are subsequently infected by the disease-causing organism.

For example, the human immunodeficiency virus (HIV) primarily infects cells of the immune system, such as T-cells, macrophages, and dendritic cells. The virus infects immune cells via the cell receptor CD4 and a co-receptor which, in primary HIV infection, is the CCR5 receptor. Both receptors are required for the virus to enter the cell where it then replicates. During the primary phase of infection, the virus replicates rapidly and infects cells throughout the body, particularly in lymphoid organs. The infection stimulates the immune system to attack the infected cells and reduce HIV levels, but the virus rapidly mutates to avoid this immune attack and so the HIV infection remains. An infected person may then remain in a symptom-free latent stage of infection for years, despite continuous replication of HIV in infected organs.

Latency may be broken if the immune system is stimulated, such as during infection by a different pathogen or by activation of CD4+ T-cells in infected lymphoid organs. HIV-infected T-cells are consequently destroyed as large amounts of virus are produced and released from the cells. Under certain conditions, apoptosis may be induced in both infected and uninfected T-cells thus further depleting the T-cell population. As the number of functional T-cells rapidly declines, immune function is compromised, and AIDS (acquired immunodeficiency syndrome) symptoms appear. The course of AIDS is characteristically described as a net change between destruction and production of CD4+ T-cells.

The course of AIDS progresses rapidly as levels of HIV (the “viral load”) increase in the bloodstream. Therapies have been focused on suppressing HIV replication by administering drug cocktails directed to highly active antiretroviral therapy (HAART). While these therapies can slow or temporarily arrest the disease, they cannot prevent further infection of the immune system by HIV. As the disease progresses, lymphoid tissues such as the thymus, bone marrow, and lymph nodes are infected and damaged, further diminishing the ability of the immune system to produce functional immune cells and to regenerate following HAART therapy.

HSC transplantation in AIDS patients is of limited use even when combined with HAART. Following transplantation, the HSC differentiate into hematopoitic progenitor cells, which give rise to several types of immune cells. Progenitor cells that give rise to T-cells migrate to the thymus, where T-cell differentiation takes place. Mature T-cells bearing CD4 and CCR5 receptors are then released from the thymus. Thus, the health and function of the thymus is critical for the formation of mature T-cells. While the HSC themselves cannot be infected by HIV, these mature T-cells will be readily infected by the virus. AIDS symptoms are temporarily diminished by the HSC transplantation as healthy immune cells are regenerated, but return as the regenerated cells become infected.

In addition, the success of HSC transplantation is highly dependent on stringent immunosuppressive therapy to prevent immune rejection of the transplanted cells. Traditionally, radiation and/or chemotherapy has been used to destroy the recipient's immune cells prior to transplanting HSC. Thus, a person whose immune system is already weakened by HIV infection is subjected to treatments that further deplete immune cells and leave the patient with no defense against subsequent infection.

### SUMMARY OF THE INVENTION

To overcome the shortcomings of the prior art, a new method of regenerating an immune system is provided. According to a general embodiment of the invention, a method of regenerating an immune system in a subject in need thereof comprises transplanting into the subject a plurality of isolated and purified hematopoeitic stem cells incapable of expressing a functional CCR5 surface receptor, wherein the transplanted cells differentiate into mature immune cells.

According to another embodiment of the invention, in addition to the general embodiment, a nutritional regimen comprising one or more compounds that improve the nutritional status of the subject and one or more anti-microbial compounds is administered to the subject. The nutritional regimen further comprises one or more anti-inflammatory compounds and one or more compounds that stimulate one or both of the growth and the function of the thymus of the subject, and such compounds are administered concurrently with and subsequent to the transplantation of the hematopoetic stem cells.

According to another embodiment of the invention, a method of treating a subject infected with HIV comprises transplanting into the subject a plurality of isolated and purified hematopoeitic stem cells incapable of expressing a functional CCR5 surface receptor, wherein the transferred cells differentiate into mature immune cells.

According to another embodiment of the invention, in addition to the general embodiment of the invention or to the method for treating a subject infected with HIV, the method further comprises transplanting a plurality of mesenchymal stem cells into the subject.

It is to be understood that both the foregoing general description and the following detailed description are exemplary, but are not restrictive, of the invention.

### DETAILED DESCRIPTION OF THE INVENTION

The invention is directed to an isolated and purified cell line of hematopoeitic stem cells (HSC) that are incapable of expressing a functional CCR5 receptor on the cell surface (“the CCR5Δ32 cells”). The invention is further directed to a method of regenerating the immune system in a subject in need thereof by transplanting CCR5Δ32 into the recipient subject. Because mature immune cells derived from CCR5Δ32 cells cannot express functional CCR5 receptors, they will be resistant to infection by HIV and other pathogens that use the CCR5 receptor to infect cells. (Agrawal et al., J. Virology 78: 2277-2287, 2004; McNicholl et al., Emerging Infectious Diseases 3: 262-271, 1997, both incorporated herein by reference). Differentiation of the CCR5Δ32 cells will regenerate the immune system, allow the regenerated immune system to function normally, and prevent subsequent infection of immune cells with pathogens that infect cells via the CCR5 receptor. In one embodiment, the method of regenerating the immune system also includes administration of a nutritional regimen to the patient that optimizes conditions for CCR5Δ32 cell transplantation.

Principal terms used in this description are defined as follows:
AIDS—Acquired immunodeficiency syndrome
CCR5 receptor—Cell surface protein utilized by HIV to infect host cells, often referred to as a coreceptor because HIV also requires CD4 receptors to infect a host cell.
CD4 receptor—Cell surface protein utilized by HIV to infect host cells.
Differentiation—Processes through which unspecialized cells acquire their mature form and function.
Express, expression—Production of a protein, i.e., a cell will “express” a protein when it synthesizes that protein; a protein, such as a receptor, is “expressed” when it is synthesized by a cell; and “receptor expression” is the synthesis of a receptor protein.
Graft v. host response—The destruction of host cells by donor cells transferred into the host.
HIV—Human immunodeficiency virus
HSC—hematopoeitic stem cell—Blood forming stem cells in the bone marrow capable of differentiating into any type of immune system cell. T-cells and B cells arise from these stem cells.
Host v. graft response—The destruction of donor cells transferred into a host by the host immune cells.
Immune cell—A cell capable of an immune response
Immune response—A response made by the immune system to a foreign substance, includes transplant rejection, antibody production, inflammation, and response of antigen specific lymphocytes to antigen.
Immune system—The bodily system that protects the body from foreign substances, cells, and tissues by producing the immune response and that includes especially the thymus, spleen, lymph nodes, special deposits of lymphoid tissue (as in the gastrointestinal tract and bone marrow), lymphocytes including the B-cells and T-cells, and antibodies.
Immunosuppression—Prevention, diminution, or delay of an immune response
Mature immune cell—cell of the immune system that can not differentiate into another cell type.
MSC—mesenchymal stem cell—Human bone marrow stromal stem cells that are pluripotent progenitor cells with the ability to generate cartilage, bone, muscle, tendon, ligament and fat tissues.
Nutraceutical—A food or naturally occurring food supplement thought to have a beneficial effect on human health.
Progenitor cell—A cell derived from a stem cell by differentiation that is capable of further differentiation into other cell types.
Regenerate—To replace by formation of new cells or tissue
Stem cell—Undifferentiated cell that is capable of continued propagation and capable of differentiating into other cell types.
T-cell—Any of the lymphocytes that mature in the thymus and are capable of recognizing specific peptide antigens through the receptors on their cell surface, also called T lymphocytes.
Transplantation—The process of transferring a donor stem cell into a recipient.
Viral load—The concentration of a virus in the bloodstream.

### The CCR5Δ32 Cell Line

The HSC cell line, CCR5Δ32, is a human hematopoeitic stem cell line derived from a human fetus that bears a 32-base pair deletion on chromosome 3 in the coding region of the CCR5 gene. The deletion is described in McNicholl, et al., Host genes and HIV: the role of the chemokine receptor gene CCR5 and its allele (Δ32 CCR5), Emerging Infectious Diseases 3: 261-271, 1997, and in U. S. Pat. No. 6,692,938, both incorporated herein by reference.

The CCR5Δ32 cells differentiate into functional immune cells that lack CCR5 receptors. Because the cells cannot produce the CCR5 receptor, pathogens, such as HIV and small pox virus, that require CCR5 receptor to infect cells cannot invade these cells and replicate within them. As a result, after CCR5Δ32 HSC are transplanted into, for example, an HIV infected/AIDS patient, T-cells that differentiate from the CCR5Δ32 HSC will be resistant to HIV infection. Over time, infected T-cells of the recipient will be replaced by resistant T-cells derived from CCR5Δ32 HSC, thereby reducing HIV infection and viral replication. As the regenerated immune system proliferates, the resistant immune cells will also help to repair and regenerate tissues and organs, such as the thymus, bone marrow, and lymphoid tissues, which have been damaged as a result of the HIV infection, thereby providing continued sources of healthy immune cells. In addition, because the regenerated immune system is derived from the donor cells, subsequent transplantations from the same source of donor HSC will not be rejected by the regenerated immune system.

### Nutritional Regimen

In one preferred embodiment of the method of treatment, a specialized nutritional regimen is administered to the recipient before, during, and after HSC transplantation to optimize conditions that help establish the CCR5Δ32 HSC in the recipient, and to lessen the trauma associated with immunosuppression. In HIV infected subjects, the nutritional regimen may additionally reduce viral load. The steps of the nutritional regimen are (1) administration of nutrients to optimize the nutritional status of the recipient, so that the transplanted cells will have a healthy environment in which to become established; (2) administration of anti-microbial compounds to remove and prevent infection in the recipient; (3) administration of neutraceuticals that reduce inflammation in the recipient to enhance the establishment of the transplanted HSC in the recipient; and (4) administration of compounds that stimulate the function and size of the thymus to allow continued propagation and differentiation of the transplanted HSC.

The nutritional regimen has three parts: the pre-transplantation phase, the days of HSC transplantation, and the post-transplantation phase. In the pre-transplantation phase and on the days of HSC transplantation, the nutritional regimen is focused on steps (1) and (2): improving the nutritional status of the transplantation recipient and eliminating any infections from the recipient. Nutrient compounds administered for step (1) may include antioxidants, such as acetyl-1-carnitine; alpha-lipoic acid; enzymes, such as Coenzyme Q10; vitamins, such as Vitamins C and E; thyroid extract; and nutraceuticals, such as undenatured whey protein, blueberry extract and resveratrol. Anti-microbial compounds administered for step (2) include natural antimicrobial agents, such as allicin, oregabiotic, colloidal silver, oil of oregano, Artemesia with citrus seed extract; and ozone.

In the post-transplantation phase, the nutritional regimen includes all four steps and is administered at selected intervals of approximately every 30 days for at least 4 months for up to one year after the transplantation procedure. During the post-transplantation phase, the compounds listed above for steps (1) and (2) are again administered and are supplemented with compounds that stimulate the thymus and suppress inflammation (steps 3 and 4). Compounds that stimulate growth and function of the thymus include fatty acids, such as alpha and gamma linolenic acid, eiocosapentaenoic acid (EPA), docosahexaenoic acid (DHA), and linoleic acid; phosphatidyl choline; glutathione; carotenoids; methyl cobalamine; thyroid stimulating agents, such as thyroid hormone and thyroid extracts; Vitamins A and D; minerals, such as zinc, calcium, magnesium, potassium, chromium, selenium, germanium, rubidium; human growth hormone; amino acids; adenosine monophosphate; and alkylglycerols such as shark liver oil. Anti-inflammatory compounds include butryn, cinnamon bark, curcumin, kaprex, RPR, quercitin, essential fatty acids, and vitamins C and E.

Therapeutic amounts of each of the compounds given are known to those of ordinary skill in the art, and, for each stage, the amount, specific compound, and method and timing of administration of each compound may be optimized for the recipient.

Pre-transplantation phase and transplantation phase

In one embodiment of the nutritional regimen, the pre-transplantation phase begins two days before the first transplantation procedure. The same regimen is also administered on the day(s) of HSC transplantation. In the pre-transplantation phase and transplantation phases, the following compounds are administered daily to the recipient via intravenous infusion:

Vitamin C, 50,000 mg per day

methocarbamol, 750 mg per day

Vitamin E, 1000 units per day

Coenzyme Q10, 400 mg per day

acetyl-1-carnitine, 3 g per day

alpha-lipoic acid, 1000 mg per day

ozone, 20 ml three times per day

mild silver protein or colloidal silver, 5-10 ml administered in 5% Dextrose in water

The following compounds are administered orally each day:

allicin, 450 mg, 3-4 times per day

artemesia, 150 mg per day

oregabiotic, 500 mg, 1-4 times per day

resveratol, 50-100 mg per day

ARMOUR® thyroid, 0.5 to 1 grain (Forest Pharmaceuticals, Inc.) that is greater than 2 mu/ml.

Post-transplantation phase

In one embodiment of the post-transplantation phase, each of the following compounds is administered at 20, 29, 61, 90, 118, and 365 days after the transplantation to stimulate the thymus.

Vitamin C, 50,000 mg, intravenously

Super Immune, 500 ml in water intravenously. Includes 25,000 mg of vitamin C, 200 mg of B6, 1 cc of B-complex, 10 cc of 10% calcium gluconate, 2000 mg magnesium sulfate, 750 mg pantothenic acid, 15 mg folic acid, 400 μg selenium, 2 cc of adenosine monophosphate or 7 cc of Glycyrrhizin, 10 cc of glutathione, 10 cc of 50 mg/ml taurine, 2 cc of either hydroxycobalamine or methylcobalamine, 2 cc of multi-trace minerals, and 5 cc of zinc.

Vitamin A, 50,000 i.u., intramuscularly

mixed carotenoids, 200,000 i.u., orally

zinc, 150 mg, orally

phosphatidyl choline, 500 mg in tandom slow intravenous push with 1500 mg glutathione

human growth hormone, 8 units, subcutaneously

adenosine monophosphate, 40 mg, intravenously

dipyridamole, 50 mg, 2-3 times/day, orally

N-acetyl cysteine, 8000 mg/day, orally

butryn, 3 times/day, orally

Undenatured whey protein, one tablespoon, 2 times/day, orally

L-glutamine, 5 g, 3 times/day, orally

L-arginine, 6 g, orally

Vitamin D3, 10,000 i.u., intramuscularly

Combined essential fatty acids, 1000 mg, 3 times/day, orally

ARMOUR® thyroid (thyroid extract) (Forest Pharmaceuticals, Inc.), 1 grain at greater than 2 mu/ml concentration, orally

methylcobalamine, 5 mg, 4 times/day, orally

folic acid, 15 mg, orally

Vitamin E, 1000 i.u., orally

alkylglycerols, (shark liver oil) 400 mg on days 90, 118, and 365, orally quercitin, 3 times/day, orally

Kaprex™ (Metagenics), 1 tablet 3 times/day, orally

Cinnamon bark, 3 times/day, orally

Curcumin, 3 times/day, orally.

### Immunosuppression

While a graft-versus-host reaction is unlikely in a transplantation of HSC that does not contain reactive T-cells, host rejection of the donor HSC cells remains probable. Host rejection of transplanted cells can occur with any non-matched allogenic graft. Therefore, it is necessary to suppress the host immune cells prior to HSC transplantation. Bone marrow transplantation recipients are generally subjected to toxic methods of immunosuppression such as chemotherapy and radiation to destroy the recipient's own hematopoietic cells. These procedures can give rise to anemia, neutropenia, and increased susceptibility to infection. Infection subsequent to severe immunosuppression can result in death of the recipient.

Less radical means of immunosuppression can be used that will allow engraftment of the donor cells, but will not destroy all of the recipient's own hematopoietic cells. Under these less severe conditions, the transplantation recipient develops “mixed chimerism,” in which the recipient's HSC co-exist with the donor HSC. In mixed chimerism, both the donor HSC and the recipient's HSC differentiate in the thymus and form dendritic cells that destroy any reactive T-cells that do not recognize the donor HSC as “self”. As a result, newly formed T-cells are tolerant of both donor and recipient antigens on immune cells, the recipient subject's immune cells will not destroy the donor cells, and subsequent cell transplantations from the same donor source will not require further immunosuppression. (Shizuru, J. A., et al., Purified hematopoietic stem cell grafts induce tolerance to alloantigens and can mediate positive and negative T cell selection. P.N.A.S. USA 97: 9555-9560, 2000; Nikolic, B., et al., Mixed hematopoietic chimerism allows cure of autoimmune diabetes through allogenic tolerance and reversal of autoimmunity. Diabetes 53: 376-383, 2004; is Donckier, V., et al., Donor stem cell infusion after non-myeloablative conditioning for tolerance induction to HLA mismatched adult living-donor liver graft. Transpl. Immunol. 13: 139-146, 2004, all of which are incorporated herein by reference).

Therefore, while immunosuppression may be accomplished by any method known to those in the art, less traumatic procedures are preferred to encourage mixed chimerism and to minimize trauma to the transplantation recipient. One preferred method of immunosuppression that also supports growth and development of the transplanted HSC is short-term treatment with mycophenolate mofetil in combination with administration of Vitamin D and co-transplantation of HSC with mesechymal stem cells (MSC). In this embodiment, mycophenolate mofetil (MMF) is administered to the recipient intravenously at a dose of 1-3 g over a period of 2-4 hours prior to transplantation on each day that an HSC transplantation is performed. Following the final transplantation procedure, a 1-3 g/day dose of MMF is administered to the recipient orally for 13 days. Vitamin D is also administered at 50,000 IU given orally on each day that MMF is administered.

MSC have immunosuppressive activity and appear to enhance the establishment of donor HSC in recipients. See K. Le Blanc, et al., Mesenchymal stem cells inhibit and stimulate mixed lymphocyte cultures and mitogenic responses independently of the major histocompatibility complex. Scandinavian J. Immunol. 57: 11-20, 2003; in A. Bacigalupo, Mesenchymal stem cells and hematopoietic stem cell transplantation. Best Practice& Research Clinical Haematology 17: 387-399, 2004; W. E. Fibbe and W. A. Noort, Mesenchymal stem cells and hematopoietic stem cell transplantation. Ann. N.Y. Acad. Sci. 996: 235-244 (2003), all of which are incorporated herein by reference.

In this embodiment, MSC may be isolated from adult human bone marrow, propagated in culture, and prepared for transplantation as described, for example, in K. Le Blanc, et al., Mesenchymal stem cells inhibit and stimulate mixed lymphocyte cultures and mitogenic responses independently of the major histocompatibility complex. Scandinavian J. Immunol. 57: 11-20, 2003; O. N. Kog, et al., Allogeneic mesenchymal stem cell infusion for treatment of metachromatic leukodystrophy (MLD) and Hurler syndrome (MPS0IH). Bone Marrow Transplantation 30: 215-222, 2002; Kern, S., et al., Comparative analysis of mesenchymal stem cells from bone marrow, umbilical cord blood, or adipose tissue. Stem Cells 24: 1294-1301, 2006; and reviewed in A. Bacigalupo, Mesenchymal stem cells and haematopoietic stem cell transplantation. Best Practice& Research Clinical Haematology 17: 387-399, 2004. MSC are also available commercially for transplantation research through stem cell banks, such as Cambrex Bio Science Rockland, Inc., Rockland, Me.

Other methods of immunosuppression that may be used include, but are not limited to, radiation, administration of chemotherapeutic agents such as cyclosporine, busulphan, cyclophosphamide, methotrexate, and administration of alemtuzumab or other antibodies. Preferably, methods of the present invention are accomplished in a way which reduces or eliminates the need for any radiation to be used on the subject.

### Transplantation of CCR5Δ32 HSC

The CCR5Δ32 HSC may be transferred by any appropriate methods known in the art, such as peripherally; by intra-bone marrow injection (e.g., Castello et al., Intra-bone marrow injection of bone marrow and cord blood cells: An alternative way of transplantation associated with a higher seeding efficiency. Experimental Hematology 32: 782-787, 2004; by any standard method of intra-thecal injection, by intrathymic injection (e.g.Trani et al., CD25+ immunoregulatory CD4 T cells mediate acquired central transplantation tolerance. J. Immunology 170: 279-286, 2003); by direct organ injection, or by other appropriate means. Combinations of different methods may be used for successive transplantations to increase the probability that the HSC will become established in the recipient.

Serial transplantations of CCR5Δ32 HSC will be performed to increase the likelihood that the transplanted cells will become established and regenerate the recipient's immune system. Immunosuppression will be required for the first serial transplantations of CCR5Δ32 HSC to promote chimerism, but may not be required for subsequent transplantations of CCR5Δ32 HSC. In one embodiment, CCR5Δ32 HSC will be transplanted each day for three sequential days. Different methods of transplantation may be employed on each day of transplantation. For example, IBMI and peripheral transplantations may be performed on the same day, or IBMI may be used for the first transplantation, and intrathymic injection or peripheral transplantation may be used for the second and third transplantations.

Subsequent transplantations of CCR5Δ32 HSC may again be made one year later if necessary. The number of transplantations and mode of administration of the CCR5Δ32 HSC may be optimized for each recipient.

Transplantation of CCR5Δ32 HSC into bone marrow (IBMI)

In one embodiment, the transplantation of CCR5Δ32 HSC may be made each day for three successive days. At least one of the three transplantations of CCR5Δ32 HSC is into bone marrow. The recipient is anesthetized using both local infiltration and light general anesthesia/analgesia procedures. A bone marrow cannula is utilized to enter the bone marrow through the sternum or other bone marrow site. Approximately 4×10⁵ to 1×10⁶ CCR5Δ32 HSC in 1 ml of a solution of 95% PBS+5% DMSO are injected into the bone marrow.

Alternative routes of CCR5Δ32 HSC transplantation

In another embodiment, thymic injection may be used as an alternative method of transplantation for CCR5Δ32 HSC. The recipient is prepared as described for IBMI. Ultrasound is used to guide a needle into the thymus, and approximately 1-10×10⁶ CCR5Δ32 HSC are injected directly into the thymus. During this surgical procedure, 2-10×10⁶ MSC in 1 ml of a solution of 95% PBS+5% DMSO are transplanted intravenously into the recipient.

CCR5Δ32 HSC may also be transplanted peripherally according to known methods. For this procedure, 7-9×10⁶ CCR5Δ32 HSC cells are administered intravenously.

Co-transplantation of MSC

In a preferred embodiment, MSC are transplanted at the same time as the CCR5Δ32 HSC cells. During this surgical procedure, 3×10⁶ MSC are suspended in 100 ml of 0.9% normal saline and transplanted intravenously into the recipient.

### Outcome Monitoring

Single nucleotide polymorphism (SNP) analysis may be used to monitor the donor: recipient blood cell ratio following the HSC transplantation to verify the formation of chimerism, i.e., that both donor and recipient immune cells are present. SNP analysis may be performed according to Harries, L., et al., Analysis of haematopoietic chimaerism by quantitative real-time polymerase chain reaction. Bone Marrow Transplantation 35: 283-290, 2005. Differentiation of donor HSC may be monitored by flow cytometry targeting CD8, CD4 and CD45RA receptors. A relative increase in CD4 and CD45RA receptors indicates HSC differentiation. Thymic T-cell output may be measured by TREC (T cell Receptor excision circles) quantification as described, for example, in Weinberg, K. et al., Factors affecting thymic function after allogeneic hematopoietic stem cell transplantation. Transplantation 97: 1458-1466, 2001.

It is expected that a functional immune system will be regenerated from the CCR5Δ32 HSC and will be maintained. The health of the recipient will improve and HIV/AIDS symptoms should diminish. While the recipient will still bear HIV, the viral load should be sufficiently diminished to prevent debilitating symptoms.

While treatment of AIDS patients is one important use for CCR5Δ32 HSC transplantation, the cells may also be useful in combating diseases caused by other pathogens that use the CCR5 receptor to invade cells. For example, CCR5-deficient mice do not develop tuberculosis when infected by Mycobacterium tuberculosis, but instead mount a protective immune response to the infection which prevents development of the disease. (Algood, H. M. S. and Flynn, J. L., CCR5-deficient mice control Mycobacterium tuberculosis infection despite increased pulmonary lymphocytic infiltration. J. Immunol. 173: 3287-3296. 2004, incorporated herein by reference). In addition, the CCR5Δ32 mutation is thought to have evolved as a protective mechanism against smallpox. (Galvani, A. P. and Slatkin, M., Evaluating plague and smallpox as historical selective pressures for the CCR5Δ32 HIV-resistance allele. P.N.A.S. USA 100: 15276-15279, 2003, incorporated herein by reference).

### EXAMPLES

1. Development and maintenance of the CCR5Δ32 cell line

According to a method for isolating and purifying the cell line, the cell line can be isolated, purified, and expanded from fetal liver tissue that is homozygous for the 32-base pair deletion in the CCR5 receptor gene. Using this method and standard cell isolation techniques, fetal liver tissue was pressed through a wire mesh sieve to separate the cells, which were then placed in culture. HSC were purified from liver cells by culturing and expanding in selective medium over two to three passages, using standard tissue culture techniques. The selective medium (“CCR5Δ32 medium”) comprised Dulbecco's modified Eagle's medium 1× with glucose and L-glutamine, 29.4% fetal bovine serum (FBS), penicillin/streptomycin, lymphocyte inhibiting factor (LIF) 20 ng/ml, basic fibroblast growth factor (FGF) 100 ng/ml,and stem cell factor (SCF) 4 ng/ml. The medium is supplemented with 4.2% non-essential amino acids, L-glutamine 2.34 mg/ml, 2-mercaptoethanol 2.22 mg/ml, and sodium pyruvate 0.22 mg/mi. The pH of the medium was substantially maintained at 7.4.

CCR5Δ32 medium was also used for propagation of the CCR5Δ32 cells. Using well-known cell culture techniques, the cells were plated at 1.8×10⁵ cells/cm² in a 75 cm² flask with a 0.2 micrometer vent cap. The cultures were maintained at 37° C. in a humidified incubator containing 95% air and 5% CO₂ and were subcultured prior to confluence.

Cells were extracted from the medium by dialysis for use or for storage. The media containing the cells was placed in MWCO 250,000 dialysis tubing and dialysed against a solution of phosphate buffered saline (PBS). For long-term storage, the cells are frozen in 95% PBS/5% DMSO and stored in liquid nitrogen.

Although illustrated and described herein with reference to certain specific embodiments, the present invention is nevertheless not intended to be limited to the details shown. Rather, various modifications may be made in the details within the scope and range of equivalents of the claims and without departing from the spirit of the invention.

1. An isolated and purified hematopoietic stem cell line comprising hematopoietic stem cells, which, upon differentiation, are incapable of expressing a functional cell surface CCR5 receptor.

2. A method of regenerating an immune system in a subject in need thereof, comprising transplanting into the subject a plurality of isolated and purified hematopoeitic stem cells, which, upon differentiation, are incapable of expressing a functional CCR5 surface receptor, wherein the transplanted cells differentiate into mature immune cells.

3. A method of regenerating an immune system in a subject in need thereof, comprising transplanting into the subject the hematopoietic stem cells of claim 1, wherein the transplanted cells differentiate into mature immune cells.

4. The method of claim 2 further comprising administering to the subject a nutritional regimen comprising one or more compounds that improve the nutritional status of the subject and one or more anti-microbial compounds.

5. The method of claim 2 further comprising administering to the subject after the transplanting step a nutritional regimen comprising one or more compounds that improve the nutritional status of the subject, one or more anti-microbial compounds, one or more anti-inflammatory compounds, and one or more compounds that stimulate one or both of growth and function in a thymus of the subject.

6. A method of treating a subject infected with HIV, comprising transplanting into the subject a plurality of isolated and purified hematopoeitic stem cells, which, upon differentiation, are incapable of expressing a functional CCR5 surface receptor, wherein the transferred cells differentiate into mature immune cells.

7. A method of regenerating an immune system in a subject infected with HIV, comprising transplanting into the subject the hematopoietic stem cells of claim 1, wherein the transplanted cells differentiate into mature immune cells.

8. The method of claim 6 further comprising administering to the subject a nutritional regimen comprising one or more compounds that improve the nutritional status of the subject and one or more anti-microbial compounds.

9. The method of claim 6 further comprising administering to the subject after the transplanting step a nutritional regimen comprising one or more compounds that improve the nutritional status of the subject, one or more anti-microbial compounds, one or more anti-inflammatory compounds, and one or more compounds that stimulate one or both of growth and function in a thymus of the subject.

10. The method of claim 2 further comprising the step of treating the subject with an immunosuppressive agent or with a regimen of radiation.

11. The method of claim 2 further comprising treating the subject with an immunosuppressive agent consisting of mycophenalate moefetil or cyclosporine A.

12. The method of claim 2, wherein the transplanting is performed by a procedure selected from the group consisting of peripheral administration, intra-bone marrow injection, intra-thymic injection, and intrathecal injection.

13. The method of claim 2, wherein the hematopoietic stem cells are CCR5Δ32 cells.

14. The method of claim 2 further comprising transplanting a plurality of mesenchymal stem cells into the subject.

15. The method of claim 14, wherein the mesenchymal stem cells are derived from bone marrow or umbilical cord blood.

16. The method of claim 2 further comprising monitoring the differentiation of the transplanted stem cells into mature immune cells.

17. A method of regenerating an immune system in a subject in need thereof, consisting of the steps of
(a) administering to the subject a nutritional regimen comprising one or more compounds that improve the nutritional status of the subject and one or more anti-microbial compounds;
(b) transplanting into the subject a plurality of isolated and purified hematopoeitic stem cells, which, upon differentiation, are incapable of expressing a functional CCR5 surface receptor, wherein the transplanted hematopoeitic stem cells differentiate into mature immune cells;
(c) transplanting into the subject a plurality of isolated mesenchymal stem cells; and
(d) subsequently administering to the subject a nutritional regimen comprising one or more compounds that improve the nutritional status of the subject, one or more anti-microbial compounds, one or more anti-inflammatory compounds, and one or more compounds that stimulate one or both of growth and function in a thymus of the subject.

18. The method of claim 17, wherein the hematopoeitic stem cells and the mesenchymal stem cells are transplanted at about the same time.

19. The method of claim 17, wherein the hematopoeitic stem cells and the mesenchymal stem cells are transplanted on each of three successive days.

20. The method of claim 17, wherein the one or more compounds that improve the nutritional status of the subject and the one or more anti-microbial compounds are administered for each of two days before transplanting the hematopoeitic stem cells.

21. The method of claim 17, wherein the one or more compounds that improve the nutritional status of the subject and the one or more anti-microbial compounds are administered on a day that the transplanting of hematopoietic stem cells step is performed.

22. The method of claim 17, wherein the step of administering a nutritional regimen comprising one or more compounds that improve the nutritional status of the subject, one or more anti-microbial compounds, one or more anti-inflammatory compounds, and one or more compounds that stimulate one or both of growth and function in a thymus of the subject is performed approximately every 30 days for at least four months and up to one year.

23. The hematopoietic stem cell line of claim 1, wherein, the hematopoietic stem cell line is from ATCC patent deposit number PTA-7998.

24. The method of claim 2, wherein the hematopoietic stem cells are derived from ATCC patent deposit number PTA-7998.

25. The method of claim 3, wherein the hematopoietic stem cells are derived from ATCC patent deposit number PTA-7998.

26. The method of claim 6, wherein the hematopoietic stem cells are derived from ATCC patent deposit number PTA-7998.

27. The method of claim 7, wherein the hematopoietic stem cells are derived from ATCC patent deposit number PTA-7998.

28. The method of claim 17, wherein the hematopoietic stem cells are derived from ATCC patent deposit number PTA-7998.

29. The method of claim 7 further comprising administering to the subject after the transplanting step a nutritional regimen comprising one or more compounds that improve the nutritional status of the subject, one or more anti-microbial compounds, one or more anti-inflammatory compounds, and one or more compounds that stimulate one or both of growth and function in a thymus of the subject.
